(19) United States
(12) Patent Application Publication
(10) Pub. No.: US 2022/0317108 A1
(43) Pub. Date: Oct. 6, 2022

(54) PETROLEUM PETROGRAPHIC FABRIC ANALYSIS FOR UNCONSOLIDATED RESERVOIRS

(71) Applicant: Julie Dee Bell, Oakville (CA)

(72) Inventor: Julie Dee Bell, Oakville (CA)

(21) Appl. No.: 17/301,480

(22) Filed: Apr. 5, 2021

(51) Int. Cl. G01N 33/24; G01N 33/28; G02B 21/36; G01V 99/00

(52) U.S. Cl. CPC G01N 33/241; G01N 33/28; G02B 21/365; G01V 99/005

(57) ABSTRACT

A systematic method of analyzing petroleum fabric for unconsolidated reservoirs is disclosed. The method includes the steps of: obtaining an oil sands sample that defines a fabric, preparing a thin section to a predetermined thickness;

setting a microscope magnification; setting a bar scale, placing the thin section under the microscope, observing and identifying elements of the fabric, categorizing the elements of the fabric, using the bar scale, based on a predetermined coarse-to-fine limit. Accordingly, first elements of the fabric are categorized as coarse components if the first elements are larger than the predetermined coarse-to-fine limit, second elements of the fabric are categorized as fine components if the second elements are smaller than the predetermined coarse-to-fine limit, and voids are categorized where the elements of the fabric are absent. The method steps further include recording, to produce data, the categorizations of the elements of the fabric and applying the data.

### BACKGROUND OF THE INVENTION

The present invention relates to unconsolidated reservoir analysis and, more particularly, to a systematic method for analyzing petroleum petrographic fabrics for unconsolidated reservoirs.

Traditional thin section analysis is based upon reservoir material being considered as crystalline rock. As a result, characterizations of unconsolidated reservoirs (i.e., tight gas, heavy oil, and bitumen sand) are inaccurately assessed. Addressing these petroleum reservoirs as crystalline rock and not as unconsolidated material can lead to incorrect conclusions being drawn for reservoir management. This often results in adverse monetary and/or environmental impacts.

Unconsolidated petroleum reservoirs are non-crystalline and are often formed from shallow burial. Therefore, the reservoir material may be compacted, but not crystalline. Often, unconsolidated reservoirs are composed of sediments, which are commonly sands, and this material differs from crystalline rock.

There are numerous problems associated with unconsolidated reservoirs, including, but not limited to, the following. (1) Sand production during recovery can be problematic. (2) The presence of clays (including clay type and spatial location) affects production and can also create environmental problems, as tailings are often made up of the clay content. (3) Heterogeneities within unconsolidated reservoirs create physical features that can alter reservoir properties, thereby restricting flow. These physical features occur on the microscale and are similar to larger scales physical features or sedimentary structures including bioturbation, laminations and others. (4) During traditional testing (such as core plug analysis, which was designed for rock analysis), unconsolidated reservoir samples collapse. (5) Petrophysical testing results can be skewed by disturbances within/around well. (6) Steam-assisted gravity drainage (SAGD) recovery well are used for in situ recovery and this method is greatly used for more environmentally friendly recovery methods.

As can be seen, there is a need for a systematic method that improves on existing methods by treating unconsolidated reservoir material according to the nature of the material instead of generalizing it as crystalline reservoir material as is done in traditional rock petrographic methods.

### SUMMARY OF THE INVENTION

In one aspect of the present invention, a method of analyzing petroleum fabric for unconsolidated reservoirs includes the steps of: obtaining an oil sands sample that defines a fabric; preparing, from the oil sands sample, a thin section to a predetermined thickness; setting a microscope magnification to a low, predetermined magnification; setting a predetermined bar scale; placing the thin section under the microscope; observing and identifying elements of the fabric; categorizing the elements of the fabric, using the predetermined bar scale, based on a predetermined coarse-to-fine limit, wherein first elements of the fabric are categorized as coarse components if the first elements are larger than the predetermined coarse-to-fine limit, wherein second elements of the fabric are categorized as fine components if the second elements are smaller than the predetermined coarse-to-fine limit, wherein voids are categorized where the elements of the fabric are absent; recording, to produce data, the categorizations of the elements of the fabric; and applying the data.

Traditional rock petrographic methods for crystalline rock use the major groupings of inter-granular and intra-granular terms to display related distribution of constituents, which historically indicate some sort of crystallization has occurred. The systematic methodology described herein takes into consideration the inherent nature of unconsolidated reservoirs (which include tight gas, heavy oil, and bitumen sands). This is an improvement on existing methods, as the systematic method described herein does not treat the reservoir constituents as crystalline material. Embodiments of the present invention provide a holistic approach for analyzing the samples by including both textural and spatial information about the reservoir constituents on a grain-to-pore scale. This scale is extremely important as it is on this scale that engineering properties, that control the reservoir flow, are located.

In accordance with the present invention, reservoir material and individual constituents viewed in thin section are considered as a whole and analyzed on the basis of heterogeneities. Microscopes used can be plain and polarized light, and also florescent. The aim of the systematic petrographic method that results from the present invention is to obtain unconsolidated reservoir characteristics to aid in determining reservoir quality. The petrographic method is based on setting a size limit at a designated magnification for the microscope and systematically and quantifiably analyzing the nature of the reservoir material or constituents and grouping the constituents into components based on their morphology.

To achieve the aim of this petrographic method, reservoir constituents are observed in thin section at 10× magnification and separated into components based on setting a coarse-to-fine size limit at 20 microns. Reservoir constituents are grouped into coarse, fine, and void components with petroleum grouped according to its size, as per to be defined terms. The unconsolidated reservoir material is viewed solely as a geological material and specifically as a petroleum reservoir material, not as a soil and therefore does not include genesis. As the primary grain size of unconsolidated reservoirs are mostly sand-sized, basic reservoir characteristics can be obtained using this method and determining partial fabrics.

Constituents that make up unconsolidated reservoirs are generally randomly oriented. In going from three-dimensional (3D) to two-dimensional (2D) and observing objects under the microscope, it should be noted that samples are not generally always cut through their largest diameter. Therefore, the size of the constituent observed in thin section is not equal to the size observed in 3D and, in general, the constituent is smaller. As a result of random plane selection through grains with an actual diameter of 1 millimeter (mm), the average mean will be 0.785 mm. It is anticipated that smaller objects might be closer to their observed size and, although this might be the case, the fine component cannot be resolved individually.

Advantageously, the size of constituents with a coarse-to-fine size limit of 20 microns determined at 10× magnification ensures that the size of the constituents most closely corresponds to the Wentworth grain size scale.

Therefore, the fine component are silt and clay sized. However, due to the effect of going from 3D to 2D, very small grains might be included in the fine component. It should be noted that limitations occur in other reservoir characterization studies such as size analysis determined by Particle Size Distribution (PSD) as maximum grain lengths may not measure, especially elongate grains thus skewing results.

During traditional testing, such as core plug analysis (which was designed for rock analysis), unconsolidated reservoir samples collapse during testing.

These and other features, aspects and advantages of the present invention will become better understood with reference to the following drawings, description and claims.

### BRIEF DESCRIPTION OF THE DRAWINGS

FIG. 1 is a flow chart of an embodiment of the present invention;

FIG. 2 is a representation of an exemplary first thin section of the embodiment of the present invention, showing exemplary coarse components, fine components, and voids, with the scale bar of the representation being 75 microns;

FIG. 3 is a representation of the first thin section of the embodiment of the present invention, showing an example of petroleum, with the scale bar of the representation being 75 microns;

FIG. 4 is a representation of a photomicrograph of coarse grain granite with a micron scale bar overlying for scale to determine coarse-to-fine ratio;

FIG. 5 is a representation of the first thin section of the embodiment of the present invention, showing a coarse-to-fine distribution, with the scale bar of the representation being 75 microns;

FIG. 6 is a representation of the first thin section of the embodiment of the present invention, showing an example of a fabric unit, which is all the quartz grains (labelled as Q) which are larger than 20 microns that are grouped together, with the scale bar of the representation being 75 microns; and

FIG. 7 is a representation of a photomicrograph taken on the edge of a trace fossil, which is an example of bioturbation creating reservoir heterogeneities.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, one embodiment of the present invention is a method of analyzing petroleum fabric for unconsolidated reservoirs, with the method including the steps of: obtaining an oil sands sample that defines a fabric; preparing, from the oil sands sample, a thin section to a predetermined thickness; setting a microscope magnification to a low, predetermined magnification; setting a predetermined bar scale; placing the thin section under the microscope; observing and identifying elements of the fabric; categorizing the elements of the fabric, using the predetermined bar scale, based on a predetermined coarse-to-fine limit, wherein first elements of the fabric are categorized as coarse components if the first elements are larger than the predetermined coarse-to-fine limit, wherein second elements of the fabric are categorized as fine components if the second elements are smaller than the predetermined coarse-to-fine limit, wherein voids are categorized where the elements of the fabric are absent; recording, to produce data, the categorizations of the elements of the fabric; and applying the data.

As mentioned above, historically, rock petrographic methods have been used to determine the characteristics of unconsolidated reservoirs, leading to shortfalls due to trying to fit a rock-based methodology to a vastly different reservoir material. The systematic, quantifiable approach described herein provides a systematic thin section method for the characterization of unconsolidated petroleum reservoirs based on grain to pore reservoir constituents in a spatial relation to each other at a predetermined magnification which create patterns of fabric units.

Specifically, an embodiment of the present invention is a systematic method based on grouping reservoir constituents observed in thin section into size and designating components based on morphology at a pre-determined coarse-to-fine size limit of 20 microns at 10× magnification. This method considers the sample holistically and it includes all constituents in relation to each other.

In accordance with the present invention, the petrographic method described herein does not make the, often faulty, assumption that ‘all petroleum reservoir material is crystalline’. Rather, the present invention provides a systematic method to obtain characteristics of unconsolidated reservoirs (including tight gas, heavy oil, and bitumen sands), by assessing the nature of reservoir constituents. Reservoir material and individual constituents viewed in thin section must be considered as a whole and analyzed on the basis of heterogeneities. This method provides both textural and spatial information about the reservoir constituents on a grain to pore scale.

The present invention addresses problems associated with unconsolidated reservoirs in many ways. First, the present invention includes the observation of partial fabrics of reservoir constituents, such that samples disturbances within/around well can be explored to better understand skewing of petrophysical testing results. Second, the present invention includes the exploration of heterogeneities in a reservoir by comparing areas of similar material that contain heterogeneities against areas where these variations from heterogeneities do not occur. Heterogeneities can be bioturbation, laminations and other physical features noted on larger scales as sedimentary structures but also occur on the grain to pore scale where fluid flow occurs. Third, the present invention provides a better understanding of the fine component including the nature of the silts and clays along with their spatial locations. This improves on the grain mount analysis currently used in oil sands by observing domains of the finer constituents (including silts and clays) which are washed away during analysis. Fourth, the present invention includes the determination of important reservoir characteristics by defining reservoir constituents which are the reservoir archaeology. Fifth, the present invention confirms analysis from traditional testing, including particle size distribution (PSD), by comparing results. Sixth, the present invention can be combined with traditional testing such as core plug analysis in obtaining reservoir characteristics. Using the teachings of the present invention, reservoir material from ‘unconsolidated reservoirs’ can be consistently compared with equal footing so that problems such as heterogeneities can be compared. Further, case studies can be used to history match reservoirs.

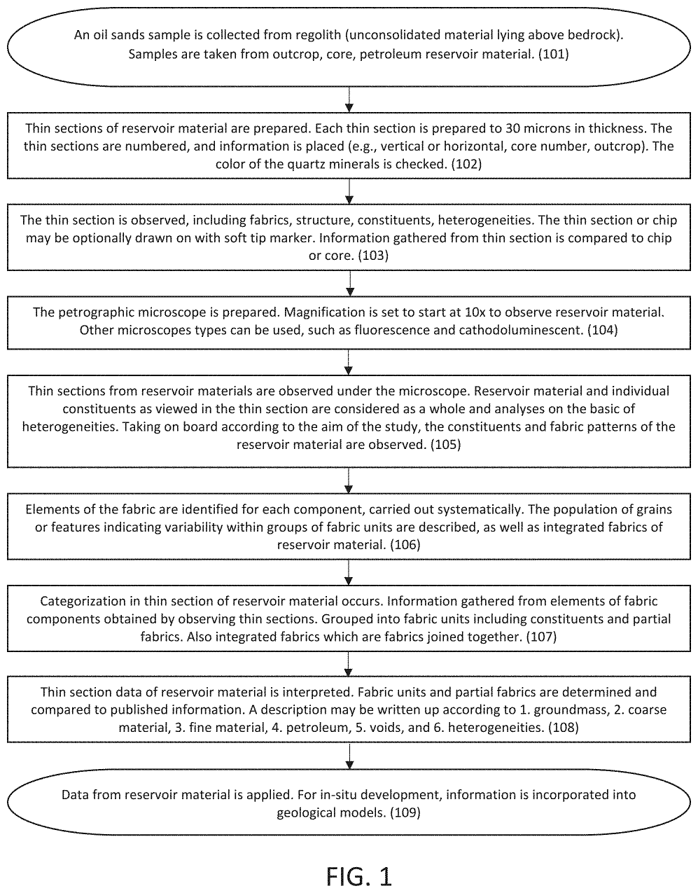

Making reference to FIGS. 1-7, the steps of an embodiment of the present invention related to the analysis of all thin sections, analysis of each individual thin section, and analysis of each individual thin section under the microscope and may be generalized as follows: 1. Observation; 2. Identification; 3. Categorization; 4. Interpretation; and 5. Application.

### Terms and Definitions:

The observation, identification, categorization, interpretation, and application of unconsolidated reservoir characteristics as viewed in thin sections in accordance with the following terms and definitions.

a) Constituents Geological factors, such as grains and pores, that are the reservoir's individual building blocks.

### b) Coarse Components

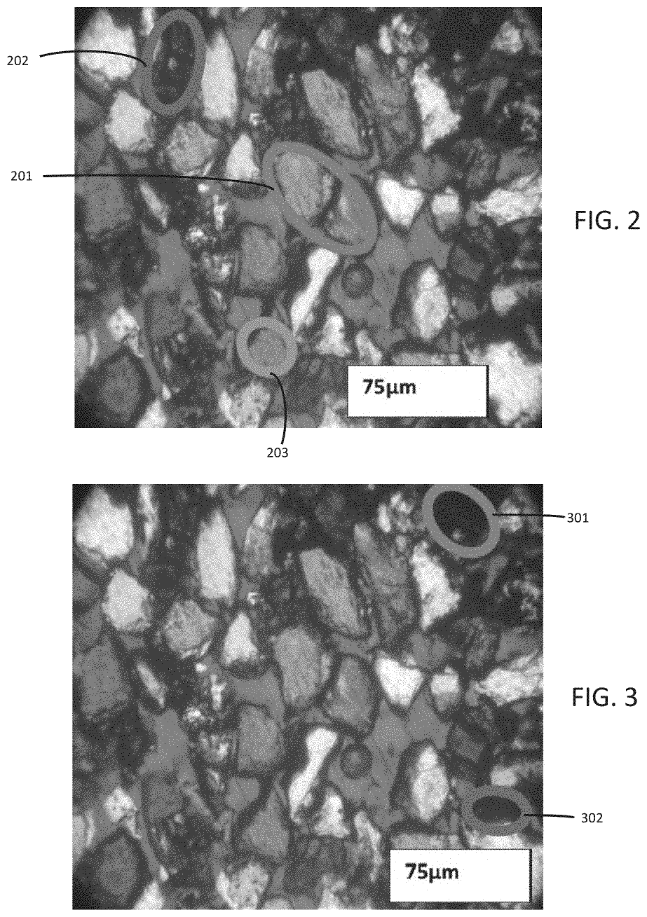

Coarse components are fabric units in which constituents are greater than the predetermined c/f limit set for the study and should be classified directly by a microscope. This component includes single mineral grains or primary grains, compound mineral grains or rock fragments, inorganic residues such as shells, and anthropogenic elements. With reference to FIG. 2, an exemplary coarse component is denoted by reference number 201.

### c) Fine Components

The fine components cannot be resolved individually by a microscope and must be viewed in groups or domains based on spatial location in respect to coarse components and voids. Clays are beyond individual observation with a petrographic microscope. With a size limit set at 20 microns at 10× magnification, anything smaller is considered silt or clay and the very fine sand according to the Wentworth scale. With reference to FIG. 2, an exemplary fine component is denoted by reference number 202.

### d) Voids

In thin section, voids are spaces not occupied by constituents and, although they are 3D objects are viewed as 2D. With reference to FIG. 2, an exemplary void is denoted by reference number 203.

### e) Petroleum

Petroleum can be heavy oil, bitumen, or natural gas. Heavy Oil which has a viscosity of between 20 to 10 degrees API Bitumen has very complicated chemical and physical properties as it consists of several chemical elements and a viscosity of below 10 degrees API Bitumen is either a coarse or a fine component according to its size as observed under the microscope. Natural gas occurs in unconsolidated reservoirs for example tight gas reservoirs. With reference to FIG. 3, reference number 301 corresponds to petroleum as a coarse component and reference number 302 corresponds to petroleum as a fine component.

### f) Others

Anything other than coarse component, fine component, bitumen/petroleum, and voids falls into this category.

### g) Elements of Fabric

The characteristics that are used to describe the constituents are the ‘element of fabric’. Each component has its own ‘elements of fabric’ criteria.
Coarse components include composition, size, shape, and sorting, along with weathering and alteration.
Fine components include color, orientation, frequency, and organization.
Petroleum include in fabric descriptors are color, size, shape, frequency and arrangement within both the coarse and fine components.
Voids can be classified into three major groups based on their size and shape.
1) Micropores, measuring 2 micron (μm) or less in width (although beyond the scope of this method); mesopores with widths between 2 and 50 μm; and macropores with widths larger than 50 μm.
2) Types of voids are characterized according to shape or morphology and their association to the other components, coarse or fine components. There are packing voids, vesicles, channels, chambers, vughs, and planes. There are three types of packing voids: simple packing voids, located between grains with no fines present; compound packing voids, located between non-accommodating aggregates of fine material; and complex packing voids, located between mixed aggregates of fines and grains. Other types of voids include: vesicles, rounded voids with smooth walls caused by air or perhaps gas; channels, tubular in shape with smooth walls; chambers, equidimensional and interconnected by channels; vughs, more or less equidimensional irregular voids with smooth or rough walls and generally not interconnected; and planes, which are flat voids with at least one sharp end.
   h) Coarse-to-Fine (c/f) Limit

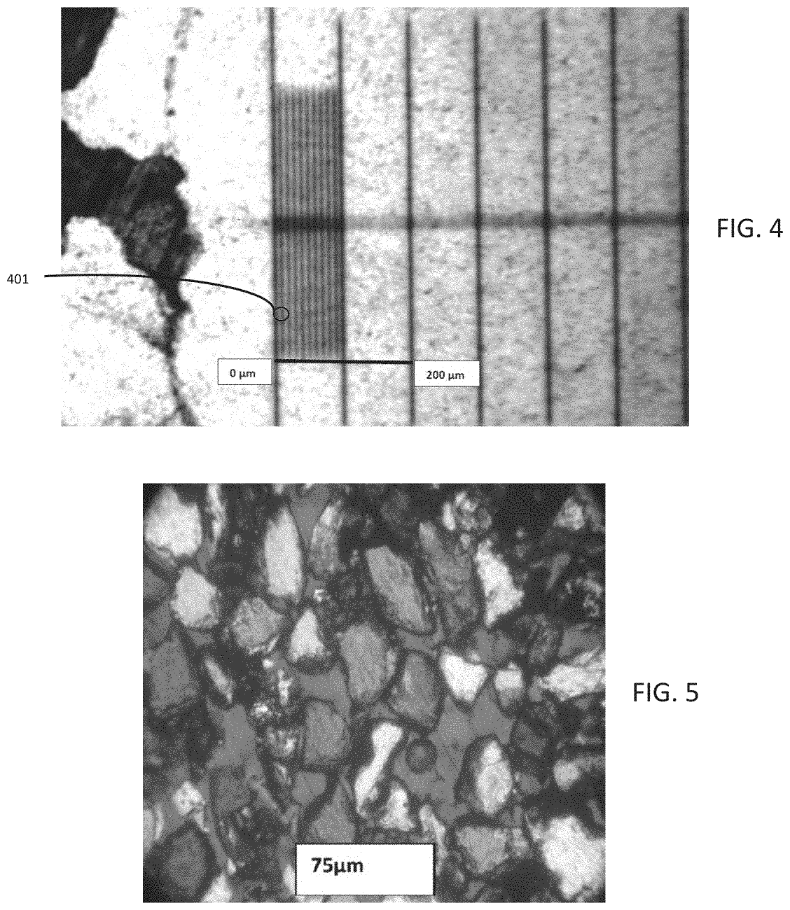

As shown in FIG. 4, or this method, the coarse-to-fine (c/f) limit is defined at a coarse-to-fine size limit of 20 microns at 10× magnification. This differs to the traditional definition of a coarse-to-fine (c/f) limit which is a floating size limit based on overall size of constituents, with no limitations on nature or complexity of the constituents. As the primary grain of unconsolidated reservoirs are mostly sand sized, basic reservoir characteristics can be obtained using this size limit. The size of constituents with a coarse-to-fine size limit of 20 microns (generally denoted by reference number 401, which is an area circled that corresponds to 20 microns) determined at 10× magnification ensures that the size of the constituents most closely correspond to the Wentworth grain size scale although limitations occur when reservoir constituents go from 3D to 2D.

### i) Coarse-to-Fine Distribution

As shown at least partially in FIG. 4, once the c/f limit is set, the c/f distribution can be determined, whereby constituents are grouped into coarse and fine components.

### j) Patterns

Pattern defined is the spatial orientation of fabric units. The spatial distribution and orientation of individual fabric units in relation to smaller fabric unites and associated pores is the coarse-to-fine related distribution. Refer to FIG. 5 as an example that illustrates this.

### k) Fabric Units

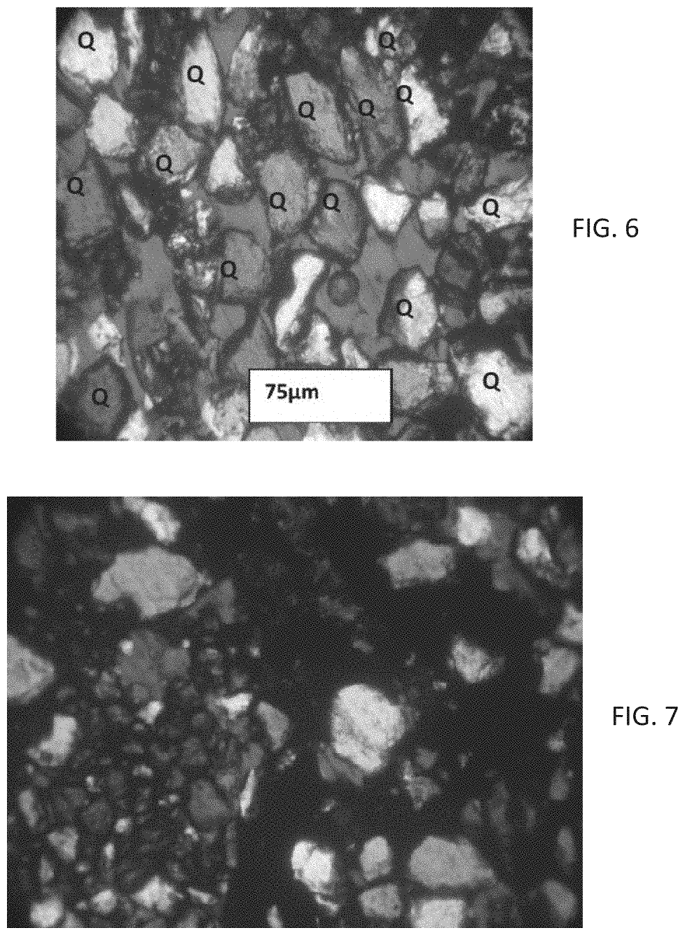

The individual constituents form the fabric units. For example, as shown in FIG. 6, a fabric unit are all quartz grains within the sample observed. How individual constituents of components come together is the fabric units.

### l) Partial Fabrics

The combinations of identical fabric units at a given scale, whether interconnected or not. Refer to FIGS. 6 and 7 as examples that illustrate this.

All thin sections and individual thin sections are analyzed: (1), to determine if any physical features such as bedding and lamination that are on a larger scale than microscopic are present; (2) to determine if any artefacts are present and note the areas where they occur; and (3) to identify any potential problems before performing the microscopic analysis.

In thin section under the microscope, a size limit of 20 microns is set at 10× magnification. Constituents are grouped together to form components and the relationships between the components based on size and determined based on morphology. The individual constituents of the components are observed and identified as they occur spatially in the thin section as observed under the microscope. Coarse components are larger than 20 microns. Fine components are smaller than 20 microns. Voids are areas where constituents are absent. Petroleum is classified either as a coarse component or a fine component according to its physical size.

For all thin sections and for individual thin sections:
Reservoir material and individual constituents as viewed in thin sections must be considered as a whole and analyzed on the basis of heterogeneities.
Physical features, artefacts and any potential problems are detailed from the thin sections. Any information is recorded and incorporated prior to thin sections analyzed under the microscope.

Under the microscope, taking on-board any physical features, artefacts and any potential problems observed from the thin section are then observed under the microscope. Plain, polarized light to fluorescent light microscopes are used to confirm petroleum component. The steps, as previously mentioned, are summarized in more detail by the following, with even greater detail further below:

### a) Observation

Reservoir material and individual constituents as viewed in the thin section must be considered as a whole and analyzed on the basis of heterogeneities.
Using a correct scale when partitioning constituents into components based on size.

### b) Identification

Identifying the ‘elements of fabric’ for each component is carried out systematically.

### c) Categorization

Any information gathered from elements of fabric of components obtained by observing the thin sections are used to group fabric units and identify partial fabrics.

### d) Interpretation

Determining fabric units and partial fabrics.
Comparing with published information.

### e) Application

For example, incorporating information into geological models.

As explained in detail below, the method described herein includes three stages of assessment for thin sections taken from unconsolidated reservoirs. Prior to the assessment in these stages, an oil sands sampled is first collected from regolith (Step 101). Samples are taken from outcrop, core, and/or petroleum reservoir material. Then, thin sections of reservoir material are prepared. Each thin section is prepared to 30 microns in thickness. The thin sections are numbered, and information is placed on them (e.g., vertical or horizontal, core number, outcrop) (Step 102). Next, the thin section is observed, including fabrics, structure, constituents, heterogeneities. The thin section or chip may be optionally drawn on with soft tip marker. Information gathered from thin section is compared to chip or core (Step 103). Step 103 allows for comparison between mesoscale and microscale, and generally corresponds to Stages 1 and 2 of assessment as detailed below. Prior to Stage 3, the petrographic microscope is prepared. Magnification is set to start at 10× to observe the reservoir material. Other microscopes types can be used, such as fluorescence and cathodoluminescent (Step 104). Below is a detailed breakdown of all systematic procedures to be followed in each of the three aforementioned stages.

### Stage 1: Visual Observation of the Entire Set of Thin Sections; Conducted Prior to Microscope Observation

The aim is to determine any physical features, such as bedding and lamination, that are on a larger scale than microscopic so that during observation under the microscope any artefacts, physical features and any problems are noted in relation to the areas where they occur in thin sections.

### Observation

a) Before observing under the microscope, ensure all thin sections were numbered properly during sample preparation. Thin sections are numbered to denote the sample number, then a hyphen, followed by thin section number e.g., (sample) 1—(thin section) 2.
b) Undisturbed samples from which thin sections are produced are kept and numbered in the same way as the thin section.
c) Thickness of the thin sections are measured and recorded.
d) The thin sections are cleaned to remove any dust or grime.
e) Thin sections are observed in together as a collection denote any areas with any potential problems.
f) Artefacts, areas not well prepared, any smearing of the sample etc. are denoted on the thin sections.
g) Spatial arrangements of coarse, fine, and voids components and where they are in relation to any physical features (such as sedimentary structures.) in the thin sections.
h) Location of petroleum components are noted in thin sections in relation to any physical features such as sedimentary structures.

### Identification

a) Spatial arrangements of obvious coarse, fine, voids and petroleum are recorded in the thin sections.
b) Any physical feature is recorded together with how it fits into the thin sections.
c) Locations of coarse features larger than coarse sand on the Wentworth scale are identified and recorded.
d) If obvious (without the aid of a microscope) the nature of the coarse fine and void components is recorded by identifying and recording elements of fabric.

### Categorization

a) Areas that affected by artefacts are discarded.
b) Fabric units are grouped together and recorded.
c) Partial Fabrics are grouped together and recorded.

### Interpretation

a) Any physical features are interpreted and how thin sections fit into these features.
b) Sedimentary structures are interpreted and how thin sections fit into these features.
c) Heterogeneities are interpreted and how thin sections fit into these features.

### Application

a) Carrying these processes out on all thin sections and individual thin sections create a check and balance.
b) Details are included in report and checked with results of all thin sections.
c) Relevant details of physical features, sedimentary structures, and heterogeneities are included in report and/or geomodels.
   Stage 2: Visual Observation of Each Individual Thin Section; Conducted Prior to Microscope Observation The aim is to determine any physical features, such as bedding and lamination, that are on a larger scale than microscopic, any artefacts and note the areas where they occur. Any potential problems are determined before the under the microscope analysis.

### Observation

a) The thin section is cleaned to remove any dust or grime.
b) Thin section is observed in its entirety to denote any areas with any potential problems.
c) Artefacts, areas not prepared well, any smearing of the sample etc. are denoted on the thin section where the if artefacts are located.
d) Spatial arrangements of coarse, fine and voids components and where they are in relation to any physical features (such as sedimentary structures.) in the thin section.
e) Location of petroleum components are noted in thin section in relation to any physical features such as sedimentary structures.

### Identification

a) Spatial arrangements of obvious coarse, fine, voids and petroleum are recorded in the thin section.
b) Any physical feature is recorded and where it is located in thin section.
c) Locations of coarse features larger than coarse sand on the Wentworth scale are identified and recorded.
d) If obvious (without the aid of a microscope), the nature of the coarse fine and void components is recorded by identifying and recording elements of fabric.

### Categorization

a) Areas that affected by artefacts are discarded.
b) Fabric units are grouped together and recorded.
c) Partial Fabrics are grouped together and recorded.

### Interpretation

a) Any physical features are interpreted and how the thin section fit into these features.
b) Sedimentary structures are interpreted and how the thin section fit into these features.
c) Heterogeneities are interpreted and how the thin section fit into these features.

### Application

a) Carrying these processes out on all thin sections and individual thin sections create a check and balance.
b) Details are included in report and checked with results of all thin sections.
c) Relevant information recorded are placed into geomodel(s).

### Stage 3: Microscopic Observation of Each Individual Thin Section Under the Microscope

### Observation (Step 105)

a) The thin section is cleaned to remove any dust or grime.
b) Magnification for this method is set at low magnification such as 10×.
c) Bar scale is set for a 20 micron size for grouping of components.
d) The thin section is placed on the microscope stage and completely skimmed over to assure that no artefacts are observed in thin section i.e., plucked grains, stressed grains, strain cracked grains. Areas that have artefacts are noted, circled, and not included in the study.
e) Analysis starts at the upper left and working across the thin section until the area is completely observed. Repeated to cover the entire thin section.
f) Reservoir constituents are observed within the thin section as a whole under the microscope and in relationship to each other in respect to predetermined size limit of 20 microns.

### Identification (Step 106)

a) Reservoir constituents are identified in thin section and separated into coarse, fine, void components based on setting a coarse-to-fine size limit 20 micron.
b) Identification of constituents observed in thin section is carried out at a 10× magnification.
c) Coarse components are larger than 20 microns and elements of fabric are recorded.
d) Fine components are smaller than 20 microns and elements of fabric are recorded.
e) Voids are areas where constituents are absent and elements of fabric are recorded.
f) Petroleum including heavy oil and bitumen is classified either as a coarse component or a fine component according to its physical size.
g) When particular constituents/components are not observed, it is reported as not observed instead of absent.
h) The quality of the results of this method are reliant upon high quality thin sections.

### Categorization (Step 107)

a) Fabric units are identified which is how individual constituents come together to form fabric unit i.e., all quartz grains grouped together.
b) Petroleum fabric units occur on both the fine and coarse components.
c) Partial fabrics of coarse, fine, void components are determined. This method concerns only partial fabrics as a coarse-to-fine size limit 20 micron at 10× magnification.

### Interpretation (Step 108)

a) Physical features that would alter reservoir properties and restrict flow on the microscale.
b) Disturbances within/around well that skew petrophysical testing results can be explored.
c) Heterogeneities characterized that create physical features which alter reservoir properties restricting flow.
d) Fabric units to characterize reservoir architecture, especially the fine domains and importantly clays.
e) Partial fabrics determined to characterize reservoir architecture on a grain to pore scale (carried out systematically) to determined reservoir quality.
f) Determine reservoir architecture on a grain to pore scale to characterize partial fabrics for carbon capture and storage studies.

### Application (Step 109)

a) Reservoir characteristics can be included in geomodels.
b) Put partial fabric about reservoirs into databases to establish case study so that history matching that can be used in risk analysis studies.
c) Identify and address problem/s associated with unconsolidated reservoirs such as:
i. understanding sand production in unconsolidated reservoirs.
ii. exploring disturbances within/around well skewing petrophysical testing results.
iii. determining the presence of clays in the reservoir including clay type and spatial location of clays.
iv. exploring heterogeneities which create physical features that alter reservoir properties and can restrict flow. These physical features occur on the microscale and are similar to larger scales sedimentary structures features including bioturbation, laminations and others.
v. Combining with traditional testing such as core plug analysis and particle size distribution to confirm testing results.

The first two stages can be performed in any sequence; although it is recommended that analysis of all thin sections and analysis of the individual thin section occur before the analysis of the individual thin section under the microscopic.

The grouping order of coarse, fine and void components in the stages of categorization, interpretation and application. Although the reservoir constituents are observed and identified as they occur spatially in thin section observed under the microscope starting at the upper left working across the sample and as area is completed, moved down the thin section and repeated for the entire thin section. During categorization, interpretation and application, the components can be partitioned into any component first (e.g., voids followed by fine and coarse—or—coarse followed by fines and voids.

Applications of the present invention have many benefits (as previously described). In summary, the data determined may be combined with other methods, input into geomodels (such as petroleum software, e.g., PETREL™) and taken into a larger model), used to explored heterogeneities, and used to make decisions on the overall reservoir quality. It should be noted that any computer used in conjunction with the present invention includes at least one processing unit coupled to a form of memory. The computer includes a program product including a machine-readable program code for causing, when executed, the computer to perform steps. The program product may include software (such as PETREL™) which may either be loaded onto the computer or accessed by the computer. This method can also be used to aid in the placement of in situ wells, used for reservoir characterization analysis, and for location of heterogenous zones and features that would obstruct flow in reservoirs.

It should be understood, of course, that the foregoing relates to exemplary embodiments of the invention and that the spirit and scope of the invention is set forth in the following claims.

What is claimed is:

1. A method of analyzing petroleum fabric for unconsolidated reservoirs, the method comprising the steps of:
obtaining an oil sands sample that defines a fabric;
preparing, from the oil sands sample, a thin section to a predetermined thickness;
setting a microscope magnification to a low, predetermined magnification;
setting a predetermined bar scale;
placing the thin section under the microscope; observing and identifying elements of the fabric;
categorizing the elements of the fabric, using the predetermined bar scale, based on a predetermined coarse-to-fine limit, wherein first elements of the fabric are categorized as coarse components if the first elements are larger than the predetermined coarse-to-fine limit, wherein second elements of the fabric are categorized as fine components if the second elements are smaller than the predetermined coarse-to-fine limit, wherein voids are categorized where the elements of the fabric are absent;
recording, to produce data, the categorizations of the elements of the fabric; and
applying the data.

2. The method of claim 1, wherein the predetermined thickness is thirty microns.

3. The method of claim 1, wherein the low, predetermined magnification is ten times magnification.

4. The method of claim 1, wherein the predetermined bar scale is twenty microns.

5. The method of claim 1, wherein the predetermined coarse-to-fine limit is twenty microns.

6. The method of claim 1, wherein the step of observing and identifying step comprises starting at an upper left corner of the thin section and observing across the thin section in a horizontal direction.

7. The method of claim 1, wherein the step of applying the data comprises inputting the data into a geomodel.
